SEQUENCE LISTING (5 sequences)

SEQ ID NO: 1 - protein, 1273 aa, artificial sequence
  Mutated SARS-CoV-2 Spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1273 aa, artificial sequence
  Mutated SARS-CoV-2 Spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1255 aa, artificial sequence
  Mutated SARS-CoV-2 Spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKFFFIIGLIIGLFLVLRVGIHLCIKLKHTKKRQIYTDIEMNRLGK

SEQ ID NO: 4 - protein, 1255 aa, artificial sequence
  Mutated SARS-CoV-2 Spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKFFFIIGLIIGLFLVLRVGIHLCIKLKHTKKRQIYTDIEMNRLGK

SEQ ID NO: 5 - protein, 538 aa, artificial sequence
  Moloney murine leukemia virus
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDD

(19) United States
(12) Patent Application Publication
(10) Pub. No.: US 2023/0149537 A1
(43) Pub. Date: May 18, 2023

(54) VACCINES, ADJUVANTS, AND METHODS OF GENERATING AN IMMUNE RESPONSE

(71) Applicants: Eisai R&D Management Co., Ltd., Bunkyo-ku, Tokyo (JP); Variation Biotechnologies Inc., Ottawa, Ontario (CA)

(72) Inventors: Fabian GUSOVSKY, Lexington, MA (US); Lynn HAWKINS, Dedham, MA (US); Sally ISHIZAKA, Carlisle, MA (US); David Evander ANDERSON, Newton, MA (US); Anne-Catherine FLUCKIGER, Saint Genis Les Ollières (FR)

(73) Assignees: Eisai R&D Management Co., Ltd., Bunkyo-ku, Tokyo (JP); Variation Biotechnologies Inc., Ottawa, Ontario (CA)

(21) Appl. No.: 17/995,546

(22) PCT Filed: Apr. 6, 2021
(86) PCT No.: PCT/US2021/026058

§ 371 Date: Oct. 5, 2022

Related U.S. Application Data
(60) Provisional application No. 63/005,908, filed on Apr. 6, 2020.

(51) Int. Cl. A61K 39/215; A61P 31/14

(52) U.S. Cl. CPC A61K 39/215; A61P 31/14; A61K 2039/5258

(57) ABSTRACT

Provided herein are vaccines including coronavirus antigens and E6020, as well as methods of mitigation of coronavirus infection by administering those vaccines.

### CROSS-REFERENCE TO RELATED APPLICATIONS

This application claims the benefit of priority of United States Provisional Patent Application No. 63/005,908, filed on Apr. 6, 2020. That application is incorporated by reference as if fully rewritten herein.

### BACKGROUND

### Field

Embodiments of the present disclosure relate to vaccines including adjuvants and antigens, in addition to methods of generating an immune response using those vaccines for the mitigation of infection by a coronavirus.

### Background of the Related Art

Vaccines have proven to be successful methods for the mitigation of infectious diseases. Generally, they are cost effective, and do not induce antibiotic resistance to the target pathogen or affect normal flora present in the host. In many cases, such as when inducing anti-viral immunity, vaccines can prevent a disease for which there are no viable curative or ameliorative treatments available.

Vaccines function by triggering the immune system to mount a response to an agent, or antigen, typically an infectious organism or a portion thereof that is introduced into the body in a non-infectious or non-pathogenic form. Once the immune system has been “primed” or sensitized to the organism, later exposure of the immune system to this organism as an infectious pathogen results in a rapid and robust immune response that destroys the pathogen before it can multiply and infect enough cells in the host organism to cause disease symptoms. The agent, or antigen, used to prime the immune system can be the entire organism in a less infectious state, known as an attenuated organism, or in some cases, components of the organism such as carbohydrates, proteins or peptides representing various structural components of the organism.

In many cases, it is useful to enhance the immune response to the antigens present in a vaccine in order to stimulate the immune system to a sufficient extent to make a vaccine effective, i.e., to confer immunity. Many protein and most peptide and carbohydrate antigens, administered alone, do not elicit a sufficient antibody response to confer immunity. Such antigens need to be presented to the immune system in such a way that they will be recognized as foreign and will elicit an immune response. To this end, additives (adjuvants) have been devised which stimulate, enhance and/or direct the immune response toward a selected antigen.

One historical example of an adjuvant, “Freund's complete adjuvant,” consists of a mixture of mycobacteria in an oil/water emulsion. Freund's adjuvant works in two ways: first, by enhancing cell and humoral-mediated immunity, and second, by blocking rapid dispersal of the antigen challenge (the “depot effect”). However, due to frequent toxic physiological and immunological reactions to this material, Freund's adjuvant cannot be used in humans.

Another immunostimulant with adjuvant activity that has been shown to have immunostimulatory or adjuvant activity is endotoxin, also known as lipopolysaccharide (LPS). LPS stimulates the immune system by triggering an “innate” immune response, a response that has evolved to enable an organism to recognize endotoxin (and the invading bacteria of which it is a component) without the need for the organism to have been previously exposed. While LPS is too toxic to be a viable adjuvant, molecules that are structurally related to endotoxin, such as monophosphoryl lipid A (“MPL”) have been tested as adjuvants in clinical trials. Both LPS and MPL have been demonstrated to be agonists to the human toll-like receptor-4 (TLR-4). One FDA-approved adjuvant for use in humans is the aluminum persulfate salt, which is used to “depot” antigens by precipitation of the antigens. Aluminum-based adjuvants have been used in human vaccines since 1932 and, although they have a long record of safety, their mode of action is not completely understood. It is generally believed that aluminum-based adjuvants enhance immune response by activation of dendritic cells. The two most frequently used aluminum-based adjuvants are referred to as “aluminum phosphate” and “aluminum hydroxide”, with aluminum hydroxide adjuvant being the most widely used commercially. Aluminum hydroxide adjuvant is not Al(OH)₃, but rather crystalline aluminum oxyhydroxide (AlOOH) which has a larger surface area than crystalline aluminum hydroxide. Aluminum phosphate adjuvant is actually amorphous aluminum hydroxy phosphate (Al(OH)ₓ(PO4)_{y}) in which some of the hydroxyl groups of aluminum hydroxide adjuvant are replaced by phosphate groups. The surface of aluminum phosphate adjuvant is composed of Al—OH and Al—OPO₃ groups. Although they are chemically similar, the two adjuvants have different chemical properties. They are often both referred to simply as “alum” adjuvants.

E6020 is a potent TLR-4 receptor agonist, and thus is useful as an immunological adjuvant when co-administered with antigens in vaccines. Toll-like receptors (TLRs) belong to the family of innate immune receptors, which play an important role in the activation of innate immunity, regulation of cytokine expression, indirect activation of the adaptive immune system, and the recognition of pathogen-associated molecular patterns (PAMPs). E6020 has been reported for use in combination with antigen or vaccine components, e.g., an antigenic agent selected from the group consisting of antigens from pathogenic and non-pathogenic organisms, viruses, and fungi. As a further example, E6020 has been reported for use as an adjuvant in combination with proteins, peptides, antigens and vaccines that are pharmacologically active for disease states and conditions including Staphylococcus aureus, pertussis toxin, tetanus, influenza, Chagas disease, meningococcus, HIV, cancer, chlamydia, cytomegalovirus, Leishmaniasis, and whooping cough (caused by pertussis toxin). When used as a component in a vaccine, E6020 and the antigen are each present in an amount effective to elicit an immune response when administered to a host animal, embryo, or ovum being vaccinated therewith.

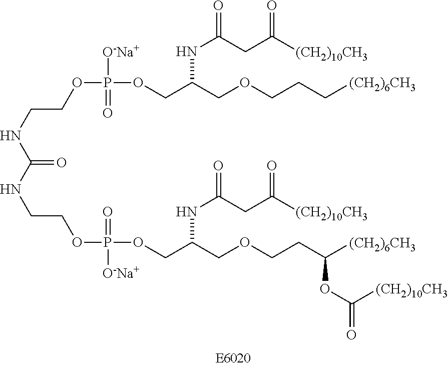

Coronaviruses are a genus in the Coronaviridae family and are pleomorphic, enveloped viruses. See S. Perlman et al., Nature Reviews Microbiology, 7:439-450 (2009). Coronaviruses contain a single stranded, 5′-capped, positive strand RNA molecule that ranges from 26-32 kb and contain at least 6 open reading frames. Coronaviruses use host proteins as part of their replication strategies. Immune, metabolic stress, cell cycling, and other cellular pathways are activated by infection. See Tang Y. et al., Front. Immunol. 11:1708 (2020).

Coronaviruses usually cause mild to moderate upper-respiratory tract illnesses, like the common cold, in people. See “Coronaviruses,” National Institute of Allergy and Infectious Disease, https://www.niaid.nih.gov/diseases-conditions/coronaviruses (accessed on Apr. 1, 2020) and Lee J. S. et al., Sci. Immunol. 5 (2020). There are hundreds of coronaviruses that affect animal species. Seven coronaviruses are known to cause human disease. Four of these coronaviruses are mild: viruses 229E, OC43, NL63 and HKU1; three of the coronaviruses can have more serious outcomes in people: SARS (severe acute respiratory syndrome), which emerged in late 2002 and disappeared by 2004; MERS (Middle East respiratory syndrome), which emerged in 2012 and remains in circulation in camels; and COVID-19, which emerged in December 2019 (a global effort is under way to contain its spread). COVID-19 is caused by the coronavirus known as SARS-CoV-2 (also known as 2019-nCoV). SARS-CoV-2 has been shown to cause mild to fatal symptoms in the human population. See Hantoushzadeh S. et al., Arch. Med. Res. 51: 347-348 (2020) and Ingraham N. E. et al., Lancet Respir. Med. 8: 544-546 (2020).

Activation of the human innate immune cells (macrophages, dendritic cells) through the binding of PAMP from SARS-Cov-2 to cell surface TLRs has been demonstrated to be a vital mediator of COVID-19 immunopathogenesis. Particularly in SARS-CoV-2 infection, major immunopathological consequences leading to death have resulted from the interaction of the SARS-CoV-2 antigens and human TLRs. Precisely, SARS-CoV-2 viral spike protein (S) binds with the extracellular domain of various TLRs including TLR1, TLR4, and TLR6, with the strongest binding with TLR4.

While treatment of specific symptoms improves survival, there are currently only a few vaccines which have been licensed for emergency use for COVID-19 prophylaxis. Thus, a need exists for new vaccines and vaccine adjuvants that are useful in provoking an immune response against COVID-19.

### BRIEF SUMMARY

Embodiments provided herein include vaccines comprising E6020 as an adjuvant and methods of generating an immune response using those vaccines for the mitigation of infection by coronavirus.

E6020 may be used as an adjuvant in vaccines directed to mitigation of nidovirales infection. Further embodiments relate to use as an adjuvant in vaccines directed to mitigation of coronavirus infection. Yet further embodiments relate to use of E6020 as an adjuvant in vaccines directed to mitigation of infection caused by the virus SAR-CoV-2 (aka COVID-19).

Embodiments include, for example, a vaccine including E6020 and an antigen related to coronavirus. Yet further embodiments relate to a vaccine including a virus like particle containing a SARS-CoV-2 spike protein and E6020.

Vaccines prepared according to embodiments presented herein may include one or more adjuvants in addition to E6020 to form an adjuvant system. For example, an embodiment of the invention includes a vaccine comprising a virus like particle containing a SARS-CoV-2 spike protein, E6020 and aluminum phosphate adjuvant. Vaccines may be formulated in a number of ways. For example, they may be formulated as a buffered solution, as an emulsion, as microparticles, or as nanoparticles (for example, as gene nanoparticles).

Vaccines prepared according to embodiments as reported herein may also include pharmaceutically acceptable additives. These include, for example, polymer additives and/or surfactant additives. Polymer and surfactant additives may be particularly useful, for example, in emulsion formulations.

Further embodiments provide methods of generating an immune response in a subject in whom an immune response to coronavirus is desired. Typically a subject is an unvaccinated human or a human who has undergone some but not all of the recommended number of doses of vaccine. In some embodiments, a subject may be a person already exposed or infected by a coronavirus, in whom a boosted immune response following vaccination is sought.

### BRIEF DESCRIPTION OF DRAWINGS

FIG. 1 depicts the detection of SARS-CoV-2 native S eVLP vaccines formulated with different adjuvants.

FIG. 2 depicts the neutralizing antibody titers from the pooled sera of C75BL/6 mice administered a SARS-CoV-2 eVLP vaccine.

FIG. 3 depicts the antibody and T cell responses of SARS-CoV-2 native S eVLP vaccines combined with different adjuvants in C75BL/6 mice.

FIG. 4 depicts the structures of SARS-CoV-2 S protein constructs and a Western blot analysis of the protein expression for each SARS-CoV-2 S protein construct.

FIG. 5 depicts the antibody titers in the sera from 20 COVID-19 confirmed convalescent patients, wherein the sera samples were separated into groups with either high or low levels of Ab binding activity to recombinant SARS-CoV-2 S.

FIG. 6 depicts humoral responses from various types of SARS-CoV-2 eVLP vaccines in C75BL/6 mice.

FIG. 7 depicts the antibody and T cell responses of different monovalent constructs of SARS-CoV-2 SPG vaccines in C75BL/6 mice.

FIG. 8 depicts the neutralizing antibody response in mice vaccinated with VBI-2902 and VBI-2901 with and without E6020.

FIG. 9 depicts the body weight changes of Syrian golden hamsters vaccinated with VBI-2902 vaccines with and without E6020.

FIG. 10 depicts the serum antibody titers of Syrian golden hamsters vaccinated with VBI-2902 vaccines with and without E6020.

FIG. 11 depicts the viral RNA levels of Syrian golden hamsters vaccinated with VBI-2902 vaccines with and without E6020.

### DETAILED DESCRIPTION

Vaccines including E6020 and methods for treatment of coronavirus infection using those vaccines will be described in detail. E6020 is present in these vaccines as an adjuvant, which is a compound that is included in a vaccine to increase the vaccine's ability to provoke, increase, and/or extend an immune response, or drive a favorable type of immune mechanism(s).

E6020

E6020 is a disodium salt of ER-804057 and is shown below:

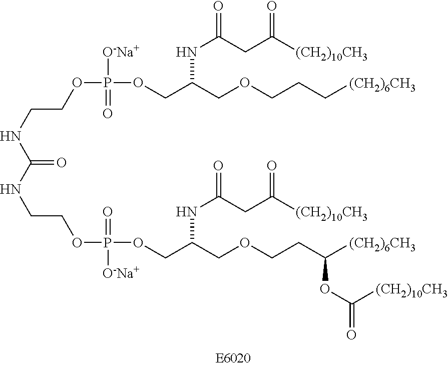

In some embodiments, the vaccines described herein include from 0.1 μg to 100 μgs, 0.5 μg to 100 μgs, 1 μg to 50 μg, 1 μg to 25 μg, 1 μg to 20 μg, 5 μg to 30 μg, 0.5 μg to 10 μg, 10 μg to 20 μg or 20 μg to 50 μg of E6020. In other embodiments, the vaccine comprises 10 μgs of E6020.

Antigens for Inclusion

Antigens are molecules that may be recognized by the immune system of a patient to generate an immune response and/or cell-mediated immunity. In embodiments reported herein, E6020 is useful as an adjuvant in a vaccine where the antigen is a nidovirales antigen, a coronavirus antigen, or a SARS-CoV-2 antigen. Antigens may be present in a vaccine in various forms. For example, an antigen may be present as purified antigen molecules (which may be, for example, proteins, multimerized proteins, protein subunits (including subunit trimers), peptides (including Ii-key peptides and locked peptides), peptides conjugated to a protein carrier, oligonucleotides, RNA (including mRNA), DNA, plasmid DNA, or polysaccharides, conjugated to a carrier or not), on live-attenuated, recombinant or inactivated-whole viruses, as dendritic cells, as antigen presenting cell vectors, as recombinant viral vectors, as adenoviral vectors, through liposomal delivery vehicles including lipoproteins or lipopolyplexes, or by a composition that includes a nucleic acid that encodes antigen.

In some embodiments, the antigen is derived from a viral or bacterial pathogen, such as an influenza or corona virus.

Vaccines described herein may comprise antigens, wherein the antigens are presented on an enveloped virus like particle (“eVLP”). eVLPs may comprise retroviral vectors that lack a retrovirus-derived genome and are therefore non-replicating. Retroviruses are enveloped RNA viruses that belong to the family Retroviridae. After infection of a host cell by a retrovirus, RNA is transcribed into DNA via the enzyme reverse transcriptase. DNA is then incorporated into the host cell's genome by an integrase enzyme and thereafter replicates as part of the host cell's DNA. eVLPs may be comprised of a structural polyprotein from a Moloney Murine Leukemia Virus (MMLV) known as a Gag protein (SEQ ID NO: 5). Expression of Gag in some host cells can result in self-assembly of the expression product into eVLPs.

The antigen may be derived from a viral or bacterial pathogen, such as an influenza virus or a corona-virus. In some embodiments, the antigen is derived from a Nidovirales virus.

In some embodiments, the antigen present within the vaccine resembles a SARS-CoV-2 spike (S) protein. The SARS-CoV-2 spike (S) protein plays a crucial role in host cell receptor binding and fusion properties leading to virus entry. See Patra R. et al., J Med Virol. 93:615-617 (2021); Aboudounya M. M. et al., Mediators Inflamm. 2021: ID 8874339; Bhattacharya, M. et al., Infect. Genet. Evol. 85: 104587 (2020). SARS-CoV-2 S proteins resemble features of class I viral proteins, wherein they are constituted of 2 subunits, 51, containing the receptor binding domain (RBD) and S2, containing the fusion entry domain. Binding of the RBD to the host cell receptor induces conformational changes resulting in activation of the protease cleavage site upstream of the fusion domain followed by release and activation of the S2 fusogenic domain. These conformational changes allow SARS-CoV-2 to infiltrate a cell and begin replication. See Khanmohammadi S. et al., J Med Virol. 1-5 (2021). SARS-CoV-2 S protein contains a furin cleavage site located at the boundary of S1 and S2 enabling rapid processing of the S protein during biosynthesis in host cells.

The antigen present within the vaccine can be a native SARS-CoV-2 S protein, a stabilized prefusion form of a SARS-CoV-2 S protein, a modified SARS-CoV-2 S wherein the TMCTD domain has been replaced with a transmembrane and cytoplasmic terminal domain of vesicular stomatitis virus G (VSV-G) or any combination thereof.

The SARS-CoV-2 spike antigen present within the vaccine may possess, for example, a protein sequence provided by SEQ ID NOs: 1-4.

In some embodiments, the vaccine may comprise from 0.1 μg to 100 μg, 0.1 μg to 50 μg, 1 μg to 25 μg, 5 μg to 25 μg or 5 μg to 10 μg of an antigen.

Vaccines that May Benefit from an E6020 Adjuvant

A number of existing or in development vaccine platforms could benefit from inclusion of E6020 as an adjuvant. These include, for example, but are not limited to those listed in Table 1.

TABLE 1

| Vaccine Platform | Organization |
|---|---|
| 2019-nCoV vaccine (protein subunit, | University of Queensland |
| molecular clamp, injectable) | |
| dendritic cell vaccine (2019-nCoV) | Beijing Dingcheng Taiyuan Biotechnology; |
| | Betta Pharmaceuticals Co. Ltd. |
| SARS-CoV-2 mRNA vaccine (COVID-19); Z- | Guanhao Biotech Co. Ltd.; Zy Therapeutics |
| VacciRNA; | Inc. |
| mRNA vaccine (LPP delivery platform, COVID- | Shanghai Yinuosi Biotechnology; Stemirna |
| 19) | Therapeutics Co. Ltd. |
| COVID-19 S-Trimer; SARS-CoV-2 vaccine | Clover Biopharmaceuticals Inc. |
| (COVID-19), recombinant 2019-nCoV S | |
| protein subunit-trimer vaccine | |
| COVID-19 vaccine (SARS-CoV-2), | AJ Vaccines A/S |
| SARS-CoV-2 mRNA vaccine (COVID-19), | CureVac AG |
| SARS-CoV-2 vaccine (oral spray, COVID-19), | MIGAL—Galilee Research Institute |
| linear DNA vaccine (2019-nCoV infection), | LineaRx Inc.; Takis Biotech |
| SARS-CoV-2 vaccine (coronavirus COVID-19 | AnGes MG Inc.; Osaka University |
| infection) | |
| recombinant viral vector vaccine (injectable, | Fudan University; ID Pharma Co Ltd. |
| 2019-nCoV infection | |
| COVID-19 vaccine (coronavirus COVID-19 | Medigen Biotechnology Corp.; National |
| infection) | Institutes of Health |
| SARS-CoV-2 peptide vaccine (li-Key, COVID- | Generex Biotechnology Corp. |
| 19), Generex | |
| live-attenuated SARS-CoV-2 vaccine (COVID- | Codagenix Inc.; Serum Institute of India Ltd. |
| 19) | |
| COVID-19 vaccine (protein subunit) | Vaxil BioTherapeutics Ltd. |
| SARS-CoV-2 vaccine (coronavirus COVID-19 | Zolovax Inc. |
| infection) | |
| live-attenuated viral vaccine (percutaneous, | Tonix Pharmaceuticals Holding Corp. |
| COVID-19) | |
| adenovirus vector vaccine (intranasal, | Altimmune Inc. |
| COVID-19) | |
| COVID-19 recombinant adenovirus vector | Greffex Inc. |
| vaccine (gene nanoparticles) | |
| SARS-CoV-2 vaccine (VLP, COVID-19) | EpiVax Inc.; University of Georgia |
| SARS-CoV-2 mRNA vaccine (COVID-19), | ModeRNA Therapeutics |
| mRNA-1273 | |
| SARS-CoV-2 vaccine (VLP, COVID-19) | Medicago Inc. |
| INO-4800; SARS-CoV-2 DNA vaccine (COVID- | Beijing Advaccine Biotechnology Co. Ltd.; |
| 19) | GeneOne Life Science Inc.; Inovio |
| | Pharmaceuticals Inc. |
| S-protein/ACE2 targeted prophylactic | Sichuan Kelun Pharmaceutical Co. Ltd. |
| polypeptide (inhalant, coronavirus COVID- | |
| 19), | |
| recombinant protein subunit vaccine (2019- | Chongqing Zhifei Biological Products Co. Ltd.; |
| nCoV infection) | Institute of Microbiology, CAS |
| Ad5-nCoV; COVID-19 vaccine (recombinant | CanSino Biologics Inc.; Institute of |
| adenovirus type-5 vector, 2019-nCoV | Biotechnology the Academy of Military |
| coronavirus infection) | Medical Sciences PLA |
| COVID-19 vaccine (protein subunit) | Sanofi Pasteur |
| BNT-162; mRNA COVID-19 vaccine (lipid | BioNTech SE; Pfizer Inc.; Shanghai Fosun |
| nanoparticle delivery system/cell-free | Pharmaceutical (Group) Co. Ltd. |
| transcription process, SARS-CoV-2 infection) | |
| DNA vaccine (SARS-CoV-2 virus) | Karolinska Institutet |
| recombinant viral vector ChAdOx vaccine | The Jenner Institute |
| (2019-nCoV infection) | |
| li-key peptide vaccine (COVID-19) | Biology Institute of Shandong Academy of |
| | Sciences; Generex Biotechnology Corp. |
| coronavirus COVID-19 infection (protein | Novavax Inc. |
| subunit vaccine, Matrix-M); protein subunit | |
| vaccine (nanoparticles, COVID-19 infection) | |
| SARS-CoV-2 recombinant adenoviral vector | Beth Israel Deaconess Medical Center; |
| vaccine (AdVac/PER.C6, COVID-19) | Biomedical Advanced Research and |
| | Development Authority (BARDA); Johnson & |
| | Johnson |
| COVID-19 recombinant adenovirus type 5 | Vaxart, Inc. |
| vector vaccine (oral enteric-coated tablet, | |
| SARS-CoV-2) (2019-nCoV infection) | |
| Monovalent or Multivalent SARS-COV-2 eVLP | Variation Biotechnologies, Inc. |
| vaccines | |

Other Adjuvants for Use in Adjuvant Systems with E6020

In some embodiments, the vaccine may comprise additional adjuvants in combination with E6020. Non-limiting examples of other adjuvants that may be useful in combination with E6020 include aluminum-based adjuvants such as aluminum hydroxide adjuvant (crystalline aluminum oxyhydroxide (AlOOH)) or aluminum phosphate adjuvant (amorphous aluminum hydroxy phosphate (for example Adju-Phos®)), an aluminum salt, chemokines, cytokines, nucleic acid sequences (particularly bacterial nucleic acid systems), lipoprotein, lipopolysaccharide (LPS), monophosphoryl lipid A, lipoteichoic acid, imiquimod, reiquimod, QS-21 or any combination thereof. Other TLR agonists may be useful in adjuvant systems with E6020. For example, other adjuvants may be agonists that activate TLR 2, 3, 5, 7, 8, 9 or a combination thereof. Use of E6020 in combination with other adjuvants may include simultaneous or sequential administration of the adjuvants in the system.

In some embodiments the vaccine further comprises from 50 μg to 50 mg, 0.1 mg to 20 mg, 1 mg to 5 mg, 3 mg to 5 mg or 0.1 mg to 1 mg of at least one additional adjuvant. In other embodiments, the vaccine further comprises 150 μg to 180 μg, such as 165 μg, of at least one additional adjuvant.

In other embodiments, the vaccine further comprises from 0.1 mg/mL to 1 mg/mL, from 0.05 mg/mL to 0.9 mg/mL, 0.5 mg/mL to 1.0 mg/mL, 0.1 mg/mL to 0.5 mg/mL, 0.1 mg/mL to 5.0 mg/mL or 0.165 mg/mL to 0.33 mg/mL of at least one additional adjuvant. In other embodiments, the vaccine further comprises 0.165 mg/mL or 0.33 mg/mL of at least one additional adjuvant. The vaccine may also comprise up to 800 mg/mL of at least one additional adjuvant. In some embodiments, the amount of the at least one additional adjuvant is based on the aluminum content within the at least one adjuvant.

Inventors of the present disclosure previously described monovalent and multivalent eVLP SARS-CoV-2 vaccines formulated with alum (U.S. Ser. No. 17/218,148) which induced a strong immune response against the SARS-CoV-2 spike protein in mice studies. These vaccines were shown to be strongly protective against COVID-19 infection in Golden Hamsters challenged with SARS-CoV-2. As described in U.S. Ser. No. 17/218,148, the eVLP SARS-CoV-2 vaccines can be formulated with a variety of different adjuvants, several of which (including E6020) were tested in mice as described in Example 4. As is shown in Example 4, the use of E6020 as an additional adjuvant to the eVLP/alum formulation resulted in a significantly stronger immune response than the use of other additional adjuvants. Furthermore, and quite surprisingly, the use of E6020 as an additional adjuvant significantly enhanced Th1-type T cell responses and a change of IgG profile with induction of significant higher IgG2 responses than seen in the formulation with the eVLP SARS-CoV-2 vaccine with alum alone or with other additional adjuvants such as mimics of AS03 and AS04. This enhanced Th1 response represents a shift in immune response toward a Th1 from Th2. The Th1 response is correlated with immunity against viral infection. Accordingly, the unexpected and significant enhancement of the Th1 response induced by the combination of the eVLP SARS-CoV-2 vaccine formulated with alum and E6020 indicates that this combination is a highly potent immunogenic composition against COVID-19.

Formulations and Modes of Administration

Vaccines including E6020 may be delivered using multiple modes of administration and multiple formulations. In addition to including coronavirus antigen and E6020, formulations typically include at least one pharmaceutically acceptable carrier.

A carrier may be, for example, a diluent, excipient, or vehicle used in administration of the E6020. Carriers may include, for example, water or saline.

Methods of administration include, but are not limited to, parenteral, e.g., intravenous, intraperitoneal, intramuscular, subcutaneous, intranasal and intrathecal. Administration can also occur systemically.

Vaccines may be formulated for parenteral administration by injection. This may include, for example, bolus injection or continuous infusion. Formulations for injection may be presented in unit dosage form, e.g., in ampoules or in multidose containers. They may include a preservative. Injectable dosage forms include suspensions, solutions or emulsions. They may be in oily or aqueous vehicles. They may contain further additives including suspending, stabilizing and/or dispersing agents. The vaccine may be in powder form for constitution with a suitable vehicle, e.g., sterile pyrogen free water, before use.

Administration by inhalation is also possible. Vaccines may be delivered, for example, as an aerosol spray presentation from pressurized packs or a nebulizer. A propellant is also used. A suitable propellant may be dichlorodifluoromethane, trichlorofluoromethane, dichlorotetrafluoroethane, carbon dioxide or other gas. A nasal spray, which is not pressurized or which is pressurized mechanically rather than chemically, can be used for intranasal administration. In the case of a pressurized aerosol, the dosage unit may be determined by providing a valve to deliver a metered amount. Capsules and cartridges may be made for use in an inhaler or insufflater. These would contain a powder mix of the compound and a suitable powder base such as lactose or starch.

Embodiments may be delivered using virus-like particles (VLPs), microparticles or nanoparticles. For example a nanoparticle may include a peptide nucleic acid oligomer conjugated to a lipid. The oligomer complexes with an antigen and/or an adjuvant, forming a nanoparticle for delivery according to one of the methods reported herein. Particle-based delivery, including microparticle or nanoparticle based delivery, may be, for example, protein-based scaffolds or matrices, lipid-based scaffolds or matrices, or polymer-based scaffolds or matrices.

In some embodiments, E6020 is formulated with enveloped virus-like particles (eVLPs) that possess a stable core of Gag protein and a lipid bilayer. eVLPs structurally resemble viruses but are much safer to administer because they lack the genetic material needed to replicate within the host. eVLPs enable repeating, array-like presentation of antigens which is a preferred means of activating B cells and eliciting high affinity antibodies. eVLPs that can be used as vaccines can be, but are not limited to, MLV-Gag eVLPs and those disclosed in U.S. Pat. No. 9,765,304 and U.S. patent application Ser. No. 17/218,148.

In some embodiments, the vaccine comprises eVLP particles comprising 0.05 mgs to 0.50 mgs, 1 μg to 50 mg, 10 μg to 10 mg, 50 μg to 5 mg or 100 μg to 500 μg of Gag protein.

In other embodiments, the vaccine comprises eVLP particles comprising an amount of antigen and an amount of Gag protein, wherein the amount of antigen is from 0.1% to 4.0% relative to the amount of Gag protein.

In some embodiments, the vaccine comprises eVLP particles comprising a of murine leukemia virus (MMLV) Gag protein. In other embodiments, the Gag protein is a MMLV-Gag protein according to SEQ ID NO: 5.

A typical treatment regimen includes administration of an amount effective or likely to lead to mitigation of an infectious agent over a period of time. That may range from a few hours to a few days, or to a few months.

Mitigation may include prevention of a disease, minimization of symptoms, or alleviation of symptoms. An effective treatment may provide mitigation for a lifetime, for decades, for years, for months, or for even a single month. The amount of mitigation may increase, decrease, or both over the time that the mitigation is in effect.

Other Additives

Other non-antigen, non-adjuvant, non-carrier compounds may be administered in or in conjunction with vaccines reported herein. For example, vaccines may include or may be administered with a separate antiviral agent, an antibacterial agent, antifungal agent, or other antibiotic. These may include, for example, oseltamivir, azithromycin, chloroqine, hydroxychloroquine, or zanamivir.

Vaccine Doses

The vaccines described herein may be administered in doses ranging from 0.1 mL to 10 mL, 0.2 mL to 5 mL or 0.3 mL to 3 mL.

### EXAMPLES

### Example 1: An Experiment to Demonstrate Adjuvant Activity would Examine In Vivo Mouse Immune Response, Comparing the Strength and Characteristics of Response to Antigen Administered with or without the Candidate Adjuvant

Example 1 is a prophetic example. In this example mice are immunized with a SARS-CoV-2 antigen, in vaccine formulations with or without E6020, in order to assess the effect of E6020 on the response to immunization. Antigens may be chosen for their potential to raise protective antibodies, cytotoxic T cell responses, or both (Grifoni A, Sidney J, Zhang Y, Scheuermann R H, Peters B, Sette A. Cell Host Microbe. 2020 Mar. 12. pii: S1931-3128(20)30166-9. doi: 10.1016/j.chom.2020.03.002.). In this example the SARS-CoV-2 antigen is recombinantly produced S protein. The S protein, or the receptor-binding domain of the S protein, can be produced by introduction of an appropriate vector into HEK cells. In this example the receptor-binding domain of the S protein, S331-524 (Tai et al., Cellular and Molecular Immunology; https://doi.org/10.1038/s41423-020-0400-4) is expressed and fused to a carrier such as the human antibody Fc to allow secretion and purification. Other approaches might include expression of full-length spike ectodomain or other subdomains of the S protein (Wang et al., https://doi.org/10.1101/2020.03.11.987958 doi: bioRxiv preprint). Alternatively an inactivated whole virus preparation might be used as antigen.

S protein sequences may be produced as monomeric subunits, or produced as fused sequences containing multimerization sequences, purification tags or sequences that allow appropriate presentation of antigenic epitopes. Various linkers or carriers of the antigenic domain can be utilized. In this example the Fc fusion is produced by inserting an appropriate S protein sequence into the pFUSE-hIgG1-Fc2 expression vector (InvivoGen, San Diego, Calif.) and expressing the resultant fusion protein by transfecting the vector into the human HEK-293 cell line. After transfection, secreted protein is recovered from the cell culture supernatant and isolated by an affinity method that selects for either the S protein (as for example with a specific anti-spike protein antibody) or the Fc sequence (e.g. using a protein A column) (ref. Tai et al. as above).

To raise a protective antibody response, groups of six to eight BALB/c mice are immunized subcutaneously with between 10 and 100 micrograms of SARS-CoV-2 S protein. Three immunizations are given at three week intervals. SARS-CoV-2 antigen is given in PBS, as an unadjuvanted control, or with a test adjuvant. E6020 is tested at doses known to augment antibody responses to other antigens, for example 1.0, 3.0 or 10 micrograms. Other adjuvants such as alum at 2.7 mg/dose, or appropriate doses of other commercially available adjuvant substances, are included as positive controls.

In all cases, responses by the groups receiving E6020 or alum or other commercially available adjuvant substances are compared to unadjuvanted groups. Control groups receiving adjuvants or carrier alone are included to confirm antigen dependence of any observed response. Similar experiments, with volumes of administration adjusted appropriately, are done looking at other routes of administration such as intranasal or intradermal. Antibody titers or neutralization titers are measured in blood, and on mucosal surfaces such as the lung or vaginal lining.

Blood samples are taken from immunized animals at two weeks after the second and/or the third immunization. Mucosal samples are taken by appropriate lavage methods. Serum or lavage are isolated and tested for anti-antigen titers of immunoglobulin using standard ELISA methods. For example, an S protein construct might be expressed that includes the sequences used to immunize, and used to coat an ELISA plate. It is important that the ELISA antigen construct not contain carrier protein sequences that are the same as those used to immunize the mice, in order to avoid measuring spurious reactivities to carrier protein that are unrelated to anti-virus responses. Examples of ELISA methods for SARS-CoV-2 S protein are given in (Nisreen M. A. Okba, Marcel A. Müller, Wentao Li, et al., medRxivhttps://doi.org/10.1101/2020.03.18.20038059) and include potential use of commercial ELISA kits. In all cases the standard ELISA procedures of coating plates with the relevant antigen, blocking the plate surface, reacting the coated plate with test serum or lavage and developing with a labeled anti-mouse immunoglobulin antibody are used. Raw ODs are plotted, or titers derived by appropriate methods.

An effective adjuvant is expected to increase the amount of anti-antigen antibody raised, cause titers to rise earlier or persist longer, or cause a shift in isotype as compared to antigen given alone. Changes in isotype response reflect differences in the cytokine patterns elicited by the adjuvant, which in turn is associated with effective protection against different types of infection. For example, IgG2a in mice is associated with an interferon-driven Th1 response that might support cytotoxic T cells and give particularly efficient anti-viral responses.

### Example 2: An Experiment to Demonstrate Adjuvant-Enhanced Elicitation of Neutralizing Antibodies Against the Coronavirus, Comparing the Strength and Characteristics of Virus Neutralizing Response to Antigen Administered with or without E6020

Example 2 is a prophetic example. Because ELISA titers may not correlate with the ability of serum antibodies to effectively protect against infection, it is helpful to measure neutralization titers elicited by immunization. In this example, mice are immunized as in Example 1, one or more times. After immunization, serum or lavage are taken as described, and are used in neutralization assays. A neutralization assay is performed by measuring the infectious potential of a pseudotyped virus which carries SARS-CoV-2 surface proteins but genetic material derived from a crippled HIV construct that cannot replicate but does express a tracking marker such as luciferase. When applied to human ACE2-expressing HEK293 cells the SARS-CoV-2 receptor binding domain allows infection and expression of luciferase by the cells. If the pseudotyped virus is exposed to neutralizing antibodies the infection is blocked and luciferase is not expressed. Such an assay is described in (Tai et al, Cellular and Molecular Immunology; https://doi.org/10.1038/s41423-020-0400-4). Specifically, pseudotyped virus is harvested from HEK293 cells cotransfected with a plasmid encoding Env-defective, luciferase-expressing HIV-1 (pNL4-3.luc.RE) and another encoding SARS-CoV-2 S protein, and pseudovirus-containing supernatants harvested subsequently.

Neutralization is assessed by incubation of pseudovirus with serially diluted mouse sera from vaccinated mice for 1 h at 37° C., followed by addition of the mixture into hACE2-expressing HEK293 cells. After an appropriate incubation the cells are lysed and the resulting supernatant is mixed with luciferase substrate and tested for relative luciferase activity, typically using a luminometer to measure light output. This assay measures production of spike-specific antibodies capable of inhibiting viral interaction with cells.

In another approach, neutralization by antibody binding to other viral proteins (as might be induced by a whole inactivated viral vaccine) is measured by a classic plaque assay in which dilutions of infectious SARS-CoV-2 are preincubated with test sera before application to a confluent culture of Vero cells. Appearance of plaques, where cells have been lysed by viral replication, will occur in the absence of neutralizing antibody. The reduction of plaque frequency in the presence of neutralization is quantified by standard means (Okba et al., medRxivhttps://doi.org/10.1101/2020.03.18.20038059). Infectious centers may also be detected and enumerated after staining the Vero culture with enzyme-tagged anti-viral specific antibody reagents.

### Example 3: An Experiment to Demonstrate Adjuvant-Enhanced Elicitation of Neutralizing Antibodies Against the Coronavirus, Using an In Vivo Challenge of Mice or Another Species in a Viral Infection Model. Groups Receiving Vaccine with or without E6020 Will be Compared for their Resistance to Infection

Example 3 is a prophetic example. To confirm authentic protective efficacy of a vaccine in the context of an in vivo infection, animals are immunized with a SARS-CoV-2 vaccine as described above and then exposed to live infectious virus in a challenge model. The animals are mice that have been engineered to allow infection by SARS-CoV-2, for example through expression of the human ACE2 receptor protein in appropriate tissues (McCray et al. JOURNAL OF VIROLOGY, January 2007, p. 813-821). Alternatively, animal species that are susceptible to infection with SARS-CoV-2 even in the absence of transgene introduction, such as ferrets or cats (Shi et al. bioRxiv preprint doi: https://doi.org/10.1101/2020.03.30.015347), are immunized with vaccine. After immunization, animals are challenged by an appropriate route, e.g. intranasally, with a dose of SARS-CoV-2 that is known to produce infection and replication. Immunization groups are compared by measuring physical symptoms (for example body temperature, oxygenation or mortality), or alternatively suppression of in vivo viral replication by the vaccine-induced immune response may be assessed by measuring viral titers in target tissues such as the lungs. This is performed by PCR, measurement of viral load in tissue homogenates (Stadler et al., Emerging Infectious Diseases⋅www.cdc.gov/eid⋅Vol. 11, No. 8, August 2005, p. 1312), or other measures such as immuno-staining for viral antigens in tissue sections. A decreased presence of virus in animals receiving vaccine adjuvanted with E6020 indicates the superior protective effect of this adjuvant.

### Example 4: Influence of Different Adjuvants on Antibody and T Cell Responses Induced by Monovalent SARS-CoV-2 eVLP Vaccines

Direct correlations between neutralizing antibody titers and T cell activity have been observed among COVID-19 patients (Ni, Immunity, 2020), suggesting that strong T cell inducing adjuvants may improve protection against SARS-CoV-2 infection. After SARS-Cov-2 infection, a preferential Th1-type response has been observed in acute and recovering patients (Weiskopf, 2020; Griffoni, Cell 2020), associated with higher levels of IgG1 Ab against the S protein (Ateyo, Immunity 2020). In contrast, a Th2-type response has been suggested to contribute to the “cytokine storm” associated with severe lung pathologies (Peeples, PNAS 2020; Roncati, 2020). In light of these results, a variety of adjuvants and adjuvant combinations were tested in combination with a native SARS-CoV-2 S eVLP vaccine for their ability to enhance neutralizing Ab production while also promoting a Th1-type response. The native SARS-CoV-2 S eVLP vaccines express the native S protein of SARS-CoV-2 (SEQ ID NO: 1). For this purpose, mimics of MF59, the adjuvant systems AS03 and AS04, and E6020 co-formulated with aluminum phosphate (Adju-Phos) were evaluated. The formulations of the native SARS-CoV-2 S eVLP vaccines with the various adjuvants are provided in Table 1.

TABLE 1
Native SARS-CoV-2 S eVLP vaccine formulations for each sample group

| | | Antigen Dose | | | Dose | | | |
|---|---|---|---|---|---|---|---|---|
| Grp | | μg | | | volume/ | Delivery | | |
| n = 10 | TA | SARS-CoV-2 | μg | Adjuvant | Animal | Route | Schedule | |
| C57BL/6 | Description | Spike/dose | Gag/dose | μg/dose* | (μL) | IP | (weeks) | Analysis |

| | | | | | | | |
|---|---|---|---|---|---|---|---|
| 1 (n = 5) | 29CH07 - | 0.1 | 50 | AS03 | 500 | immunization: | Anti-Spike |
| | Native | | | (1482.5 μg | | 0, 3 weeks | (SARS-CoV-2) |
| | Monovalent | | | DL-α- | | Bleeding: | antibody |
| | | | | tocopherol, | | POV Pre- | binding and |
| | | | | 86.25 μg | | treatment | neutralization |
| | | | | Squalene, | | P1Vd14, | titers Ab |
| | | | | 607.5 μg | | P2Vd44 (& | isotyping |
| | | | | Polysorbate 80) | | sac) | (optional) |
| 2 | 29CH07 - | 0.1 | 50 | E6020 | 500 | | Y ELISpot** |
| | Native | | | plus Alum | | | (first 5 |
| | Monovalent | | | (10 μg MPL + | | | animals/ |
| | | | | 33 μg of | | | group fresh |
| | | | | Al+++ Content) | | | spleens; |
| | | | | (Aluminum | | | remaining |
| | | | | Phosphate) | | | spleens to |
| | | | | | | | be frozen) |
| 3 | 29CH07 - | 0.1 | 50 | Modified | 500 | | |
| | Native | | | AS04 (10 μg | | | |
| | Monovalent | | | MPL + 33 μg | | | |
| | | | | of Al+++ | | | |
| | | | | Content) | | | |
| | | | | (Aluminum | | | |
| | | | | Phosphate) | | | |
| 4 | 29CH07 - | 0.1 | 50 | AS04 | 500 | | |
| | Native | | | 10 μg | | | |
| | Monovalent | | | MPL + 33 μg | | | |
| | | | | of Al+++ | | | |
| | | | | (Aluminum | | | |
| | | | | Phosphate) | | | |
| 5 | 29CH07 - | 0.1 | 50 | AS01B | 500 | | |
| | Native | | | (10 μg | | | |
| | Monovalent | | | MPL + 10 μg | | | |
| | | | | QS21) | | | |
| 6 | 29CH07 - | 0.1 | 50 | 1:1 ratio; | 500 | | |
| | Native | | | MF-59 | | | |
| | Monovalent | | | | | | |

*= proposed mouse doses of adjuvant constituents, human doses are approximately 4 to 5 times higher.

SARS-CoV-2 native S eVLPs were used to compare the effects of the adjuvants as they were less immunogenic than eVLPs expressing the prefusion form of the S protein and might better enable differences in the adjuvants to be observed. Mice received 2 IP injections of the native SARS-CoV-2 eVLPs formulated with the various adjuvants. The IgG binding titers, neutralizing antibody titers and Ab and T cell responses was assessed 14 days after the second injection.

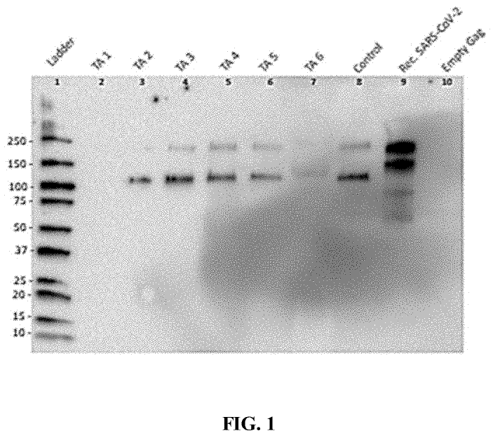

Western blot analysis of native SARS-CoV-2 eVLPs vaccines (FIG. 1) was performed using a primary antibody that is a SARS-CoV-2 (2019-nCoV) Spike RBD Rabbit Antibody (Cat #40592-T62 Sinobiological) (1/5,000 dilution). The secondary antibody is a goat anti-rabbit IgG-Fc HRP-conjugated (Bethyl, Cat #A120-11 1P-18) 1 mg/mL—1/10,000 Precision Protein Streptactin HRP conjugate (Bio-Rad, cat #161-0381) (1/10,000 dilution). TA 1 is the A503 vaccine (see Group 1 in Table 1), TA 2 is the E6020+aluminum phosphate adjuvant vaccine (see Group 2 is Table 1), TA 3 is the A504 modified vaccine (see Group 3 in Table 1), TA 4 is the A504 vaccine (see Group 4 in Table 1), TA 5 is the ASO1B vaccine (see Group 5 of Table 1), TA 6 is the MF59 vaccine (see Group 6 of Table 1), the control lane is a Native Monovalent SARS-CoV-2 vaccine from 29CH07—Post TFF/UC Pellet Filtered BDS (Lot #V20200501-nCOVID), Lane 9 is 0.025 μg of a recombinant SARS-CoV-2 (Srn T2 #DL2020APR30 0.46/mL) vaccine, and Lane 10 is 3.65 μg of 19CH102-Psot TFF/UC Pellet Filtered BDS (V20200409-EG).

The IgG binding titers of the vaccine constructs are presented in Table 2 below.

TABLE 2
IgG Binding Titers of the Native SARS-CoV-2 S eVLP vaccines

| | | | Anti SARS-CoV2 Total | | Anti SARS-CoV2 | | Anti SARS-CoV2 | |
|---|---|---|---|---|---|---|---|---|
| | Time | | IgG | | IgG1 | | IgG2b | |
| | Point | Samples | negative | | negative | | negative | |
| Groups | P0V | pooled | | average | | average | | average |
| Group 1 Native | P2Vd14 | #1 | 828,156 | 1,367,790 | 96,938 | 266,354 | 409,958 | 621,559 |
| Monovalent, | | #2 | 1,498,902 | | 374,593 | | 357,774 | |
| Covid-2/ | | #3 | 1,530,883 | | 386,931 | | 840,601 | |
| Gag = 0.1/50 | | #4 | 1,901,874 | | 360,887 | | 627,806 | |
| with ASO3 | | #5 | 1,079,134 | | 112,419 | | 871,654 | |
| Group 2 Native | | #6 | 3,187,253 | 1,657,413 | 296,242 | 178,915 | 1,281,374 | 949,624 |
| Monovalent, | | #7 | 3,013,297 | | 175,672 | | 1,656,102 | |
| Covid-2/ | | #8 | 1,364,508 | | 96,178 | | 813,822 | |
| Gag = 0.1/50 | | #9 | 1,411,128 | | 235,408 | | 630,914 | |
| with E6020 | | #10 | 2,588,920 | | 628,375 | | 935,566 | |
| plus Alum | | #11 | 1,314,792 | | 143,851 | | 762,806 | |
| | | #12 | 198,361 | | 0 | | 142,406 | |
| | | #13 | 905,748 | | 87,558 | | 532,318 | |
| | | #14 | 1,480,743 | | 81,034 | | 1,501,578 | |
| | | #15 | 1,109,384 | | 44,826 | | 1,239,350 | |
| Group 3 Native | | #16 | 1,241,791 | 762,748 | 146,279 | 96,542 | 756,649 | 672,741 |
| Monovalent, | | #17 | 565,958 | | 13,199 | | 355,302 | |
| Covid-2/ | | #18 | 1,427,824 | | 66,368 | | 1,470,150 | |
| Gag = 0.1/50 | | #19 | 995,975 | | 137,267 | | 497,541 | |
| with modified | | #20 | 532,095 | | 233,018 | | 375,635 | |
| ASO4 | | #21 | 518,825 | | 44,700 | | 581,137 | |
| | | #22 | 478,805 | | 66,723 | | 481,256 | |
| | | #23 | 81,326 | | 9,922 | | 109,066 | |
| | | #24 | 995,823 | | 148,236 | | 1,096,808 | |
| | | #25 | 789,055 | | 99,709 | | 1,003,862 | |
| Group 4 Native | | #26 | 501,977 | 716,392 | 38,811 | 81,690 | 476,032 | 336,931 |
| Monovalent, | | #27 | 1,418,675 | | 22,281 | | 147,424 | |
| Covid-2/ | | #28 | 730,790 | | 49,579 | | 357,788 | |
| Gag = 0.1/50 | | #29 | 1,326,906 | | 156,403 | | 320,280 | |
| with ASO4 | | #30 | 837,100 | | 127,327 | | 630,744 | |
| | | #31 | 651,463 | | 34,342 | | 376,865 | |
| | | #32 | 765,006 | | 263,806 | | 558,971 | |
| | | #33 | 166,217 | | 57,269 | | 56,626 | |
| | | #34 | 581,071 | | 21,104 | | 291,965 | |
| | | #35 | 184,715 | | 45,977 | | 152,620 | |
| Group 5 Native | | #36 | 1,191,558 | 769,161 | 251,887 | 180,998 | 796,244 | 456,949 |
| Monovalent, | | #43 | 346,764 | | 110,109 | | 117,655 | |
| Covid-2/ | | | | | | | | |
| Gag = 0.1/50 | | | | | | | | |
| with ASO1B | | | | | | | | |
| Group 6 Native | | #46 | 37,365 | 719,969 | 47,910 | 166,161 | 16,883 | 118,139 |
| Monovalent, | | #47 | 200,387 | | 105,304 | | 134,172 | |
| Covid-2/ | | #48 | 325,615 | | 179,750 | | 126,519 | |
| Gag = 0.1/50 | | #49 | 159,205 | | 65,391 | | 86,639 | |
| with 1:1 ratio, | | #50 | 211,532 | | 115,520 | | 82,997 | |
| MF-59 | | #51 | 364,707 | | 167,456 | | 286,230 | |
| | | #52 | 220,461 | | 140,774 | | 132,309 | |
| | | #53 | 171,082 | | 125,506 | | 65,666 | |
| | | #54 | 5,259,575 | | 567,650 | | 170,828 | |
| | | #55 | 249,758 | | 146,343 | | 79,149 | |

The virus-neutralizing titers of the vaccine constructs are presented in Table 3 below.

TABLE 3
Native SARS-CoV-2 S eVLP vaccine virus-neutralizing titers

| Sample Name | VN titer | 2Log VN titer |
|---|---|---|
| Group 1—Pooled Sera at P2VD14 | 95.4 | 6.6 |
| Group 2—Pooled Sera at P2VD14 | 95.4 | 6.6 |
| Group 3—Pooled Sera at P2VD14 | 13.7 | 3.8 |
| Group 4—Pooled Sera at P2VD14 | <=10 | <=3.3 |
| Group 5—Pooled Sera at P2VD14 | 95.1 | 6.6 |
| Group 6—Pooled Sera at P2VD14 | 16.3 | 4 |

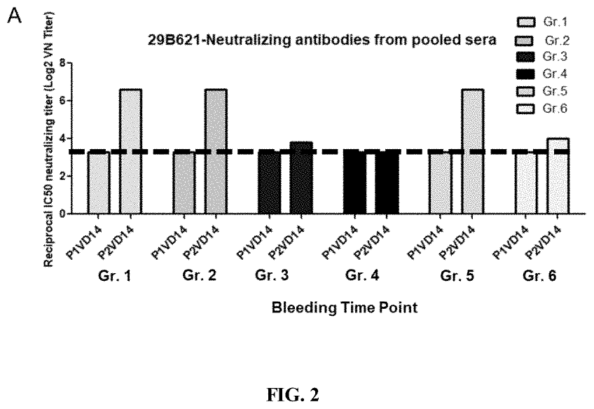

The neutralizing anti-body titers from the pooled sera of the treatment groups are depicted in FIG. 2. The neutralizing anti-body titers at P1VD14 and at P2VD14 were measured. The neutralizing anti-body titers of Group 2 were similar to those of Group 1 and higher than those of Groups 3-4. The data presented within FIG. 2 demonstrates that the use of E6020 as an additional adjuvant significantly improved the effectiveness of the native monovalent SARS-CoV-2 vaccine formulated with the aluminum phosphate adjuvant.

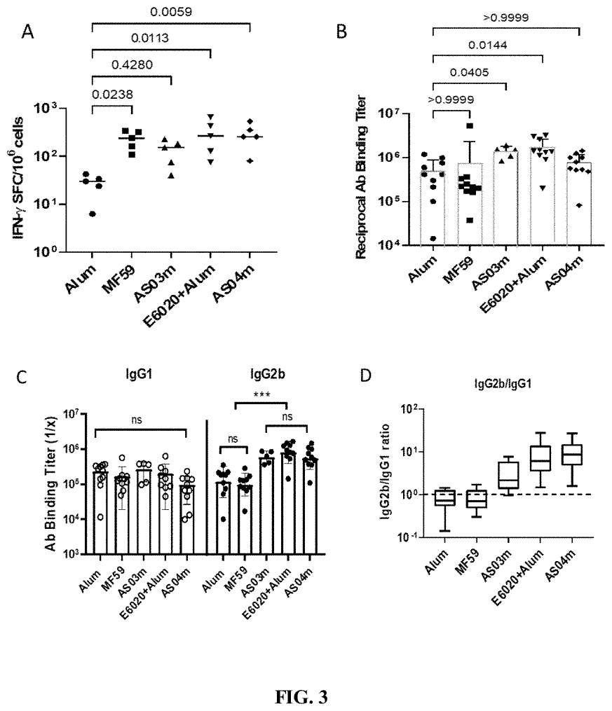

MF59 enhanced Th1-type T cell responses compared to alum (FIG. 3A) but induced similar Ab responses (FIG. 3B-C) and comparable, balanced IgG2/IgG1 ratio (FIG. 3D). In contrast, the addition of E6020 to the eVLP+aluminum phosphate adjuvant significantly enhanced Th1-type T cell responses and a change of IgG profile with induction of significant higher IgG2 than with Alum alone, demonstrating Th1 polarization of both T cell and antibody responses. Mimics of AS03 and ASO4 adjuvants also biased responses towards a Th1-type response, though to a lesser extent than E6020.

### Example 5: Contraction of SARS-CoV-2 eVLP Antigens and Vaccines

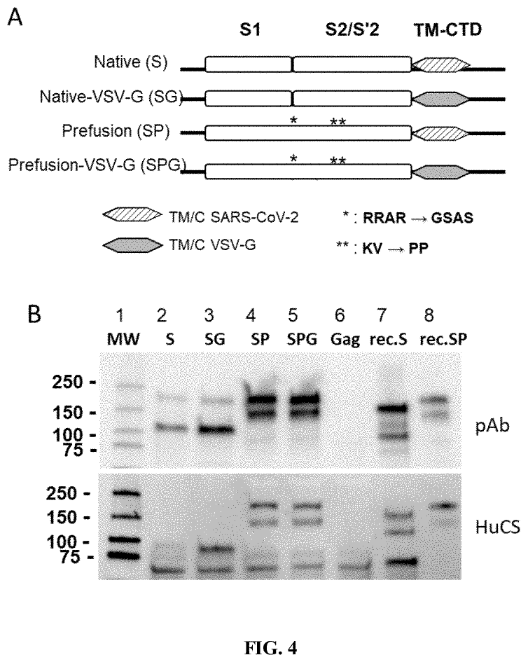

Four constructs were designed based on the S protein sequence of the SARS-CoV-2 Wuhan-Hu1 isolate and subcloned into expression plasmids for the production of eVLPs (FIG. 4) as described in U.S. patent application Ser. No. 17/218,148. One of the constructs expressed the native form of the SARS-CoV-2 S protein (S, SEQ ID NO: 1). Briefly, to obtain a stabilized prefusion form of the S protein (SP, SEQ ID NO: 2), the furin cleavage site of S was inhibited by mutation of the RRAR into GSAS and 2 proline substitutions were introduced at successive residues K986 and V987. Previous work has demonstrated that the swap of the transmembrane and cytoplasmic terminal domains (TM-CTD) of CMV gB resulted in enhanced yields and immunogenicity of the gB glycoprotein presented on eVLPs. Based on this data, two additional constructs, Native-VSVg (SG, SEQ ID NO: 3) and Stabilized Prefusion-VSVg (SPG, SEQ ID NO: 4) were designed by swapping the TM-CTD of S with that of VSV-G (FIG. 4A).

Western blot analysis of eVLPs using a polyclonal Ab directed against the SARS-CoV-2 S receptor biding domain confirmed the processing of SARS-CoV-2 S during biosynthesis in HEK293 cells as expected by the presence of the furin cleavage site in S 1/S2 (Walls, 2020) (FIG. 4B, lane 2-3). Expression of S was slightly improved by the VSV-G swap in SG, and more dramatically enhanced by the inhibition of the cleavage sites in SP and SPG (FIG. 4B, lane 4-5). Overexpression of S in the prefusion forms showed a major band at 180 Kda, the size commonly described for uncleaved 5180 Kda and an additional band around 150 Kda. The additional band around 150 Kda is reproducibly seen upon overexpression of uncleaved S, and most likely represents the S protein deprived of N-Glycosylation (Sun, 2020) that would occur because of overloading of the host cell machinery.

Quantitative analysis of protein content in eVLP preparations showed that for a similar numbers of particles, corresponding to comparable amounts of Gag protein, the amount of SARS-CoV-2 S protein was increased substantially with replacement of the TM-CTD and by use of the stabilized prefusion construct, suggesting that the density of the S protein was enhanced using the VSV-G constructs (see Table 4 below). The best yield was reproducibly obtained when producing the eVLP expressing the prefusion-VSV-G form of S, with an increase up to 40-fold more than in eVLPs expressing native S.

TABLE 4
Quantitative analysis of SARS-CoV-2 S
protein content in eVLP vaccine constructs

| | Gag total | SARS-CoV-2 S | % of S | |
|---|---|---|---|---|
| | amount | total amount | relative | Particle |
| Construct | (mg) | (mg) | to Gag | number/mL |
| Native (S) | 23 | 0.162 | 0.18 | 4.37 × 10e11 |
| Native-VSV-G | 19 | 0.50 | 0.37 | 4.37 × 10e11 |
| (SG) | | | | |
| Prefusion (SP) | 32 | 0.230 | 1.81 | 4.37 × 10e11 |
| Prefusion-VSV-G | 23 | 0.639 | 3.76 | 4.37 × 10e11 |
| (SPG) | | | | |

### Example 6: Impact of SARS-CoV-2 S Antigen Design on Neutralizing Antibody Responses

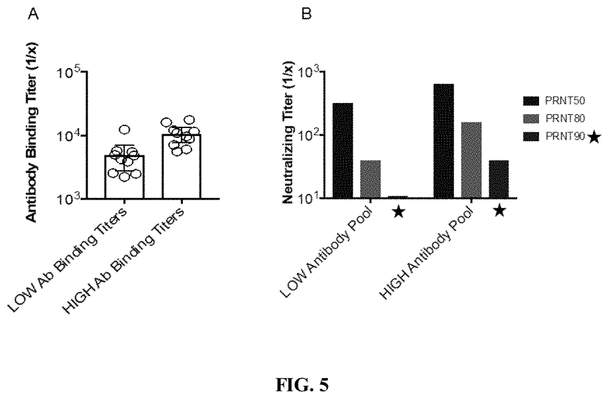

Comparison to convalescent serum is commonly used as a benchmark to help evaluate immunogenicity and potential efficacy of Covid-19 candidate vaccines. However, a wide spectrum of Ab responses can be observed in recovering patients, ranging from barely detectable to very high levels, likely influenced by time since infection and severity of disease. To enable comparison across experiments, a cohort of 20 sera from COVID-19 confirmed convalescent patients with moderate COVID-19 symptoms who all recovered without specific treatment intervention or hospitalization were obtained. The cohort was separated into two groups of 10 samples according to high or low levels of Ab binding activity to recombinant SARS-CoV-2 S (FIG. 5A). Sera from each group were then pooled and tested for neutralizing activity (FIG. 5B). As expected, the pool showing higher levels of IgG titers against SARS-CoV-2 S had the highest neutralizing activity, which was consistent with previous observations (Ni, Immunity 2020). To provide a robust benchmark with which to assess the immunogenicity of the vaccine candidates, only the high titer pooled sera was used to assess vaccine-induced animal sera.

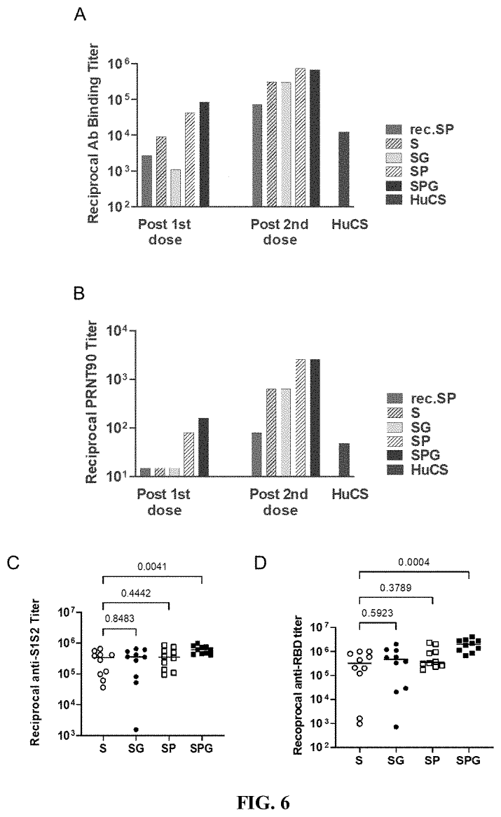

Humoral responses of the various types of SARS-CoV-2 eVLPs were evaluated in C75BL/6 mice that had received 2 intraperitoneal injections at 3 week intervals (FIG. 6). The first injection of unmodified S presented on eVLPs induced levels of anti-SARS-COV-2 S Ab binding titers similar to those in mice that received a recombinant trimerized prefusion S protein, but they were not associated with significant (90% or greater) neutralization activity as measured in a PRNT assay (FIG. 6A-B). In contrast, a significant nAb response was induced by a single injection of eVLPs expressing prefusion SP or SPG, with PRNT90 EPTs of 80 and 160 respectively that were higher than that observed with the human convalescent control pool (PRNT90 EPT of 50). All nAb responses were greatly enhanced by the second injection and reflected the responses that were observed prior to the boosting dose. Dilutions of 1/640 and 1/2560 of the pooled sera from animals immunized with native or prefusion forms of S eVLPs, respectively, neutralized 90% of the cytopathic effect of the virus. Notably, all forms of SARS-CoV-2 S presented on eVLPs induced higher antibody titers than recombinant prefusion S protein, both in the levels of total IgG and neutralization activity, after one or two injections.

Analysis of individual mice sera was performed 14 days after the second injection of eVLPs to evaluate the Ab responses against the whole S1+S2 protein or the RBD (FIG. 6C-D). All immunized mice that received eVLPs showed robust anti-SARS-Cov-2 Ab responses either against a full length S1+S2 protein (FIG. 6C) or against the RBD protein (FIG. 6D). A more homogenous response was observed in mice that received the SPG eVLPs, with all Ab EPTs above (5.6 Log 10) 400,000 against S, and above (5.8 Log 10) 650,000 against RBD.

### Example 7: eVLP Expression of a Stabilized Prefusion Form of the SARS-CoV-2 Spike Protein with E6020 Elicits Potent Immunity after a Single Dose

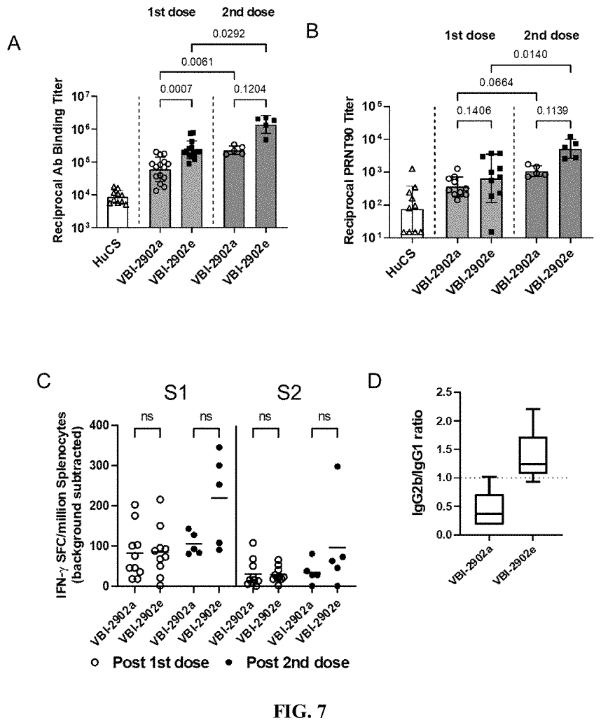

To assess the immunogenicity and potential efficacy of the vaccines disclosed herein, two embodiments possessing eVLPs expressing the stabilized prefusion VSV-G form of S (SPG from FIG. 4) were chosen because of their significantly higher production yields and greater potency. One vaccine was formulated with aluminum phosphate adjuvant (“alum”) alone, due to its extensive safety record in several approved prophylactic vaccines (“VBI-2902a”), and the other vaccine was formulated with aluminum phosphate adjuvant and E6020 (“VBI-2902-e”). Fourteen days after a single injection, mouse sera contained total anti-Spike IgG EPTs reaching geometric means of (4.8 Log 10) 54,891 with VBI-2902a and (5.2 Log 10) 258,865 with VBI-2902e that were associated with neutralizing PRNT90 titers of 365 (2.6 Log 10) for VBI-2902a and 651 (2.8 Log 10) for VBI-2902e. A second injection boosted Ab binding titers to 228,374 (5.4 Log 10) and 1,400,285 (6.2 Log 10), respectively, for VBI-2902a and VBI-2902e and nAb titers to 1,079 (3.0 Log 10) and 5,178 (3.7 Log 10) (FIG. 7A-B).

Ex vivo stimulation of splenocytes (FIG. 7C) demonstrated preferential T cell response against the 51 domain of the spike protein rather than the S2 domain. After the first injection, no differences were observed between groups that were adjuvanted with alum or with E6020+alum. The second injection of VBI-2902e induced a clear enhancement in the number of IFN-g-producing T cells in response to S1 peptides. Evaluation of IgG1 and IgG2 Ab binding titers confirmed that formulation with the TLR4 agonist E6020 resulted in an increase in IgG2 production, suggesting a Th1 polarized response (FIG. 7D).

### Example 8: Comparison of Neutralizing Antibody Responses in Monovalent and Trivalent eVLP Vaccines Formulated with and without E6020

A preclinical study was conducted to evaluate the efficacy of monovalent SARS-CoV-2 vs trivalent (eVLP constructs, with Adjuphos® or Adjuphos® and E6020 adjuvanted vaccine candidates in C57BL/6 mice. Both the trivalent and the monovalent eVLP constructs were the pre-fusion stabilized form described above. Mice were randomly assigned to 4 experimental groups as shown in Table 5 below and immunized intraperitoneally on week 0 (day 0) and week 3 (day 21) with 0.5 mL vaccine. Blood was collected on day 14 after the 1st and 2nd immunization.

TABLE 5
Vaccine formulations administered to each of the study groups

| | | | | Dose | | | |
|---|---|---|---|---|---|---|---|
| | | Antigen Dose | | Volume/ | | | |
| Group | TA | μg SARS CoV2 | Adjuvant Dose**** | mouse | Delivery | Schedule | |
| ID | description | Spike | μg | μL | Route | (weeks) | Analysis* |
| 1 | Monovalent | 0.2 | Alum phosphate | 500 | IP | Immunizatoin: | Anti-Spike (SARS-CoV-2 for |
| | SARS-CoV-2 | | (125 μg of Al+++ | | | 0.3 weeks | all groups and SARS and |
| | eVLPs | | Content) | | | Bleeding: | MERS in Trivalent groups) |
| 2 | Trivalent SARS- | 0.2 | E6020 plus Alum | | | POV (Pre- | Antibody binding, |
| | CoV-2, SRS-CoV | | (10 μg MPL + 33 μg | | | treatment | Neutralization titers |
| | and MERS-CoV | | of Al+++ Content) | | | P1Vd14 | (PRNT*** or Virions Pseudo- |
| | eVLPs | | (Aluminum Phosphate | | | P2Vd14 | particles**) |
| 3 | Monovalent | 0.2 | Alum phosphate | | | | Cross-Reactivity for |
| | SARS-CoV-2 | | (125 μg of Al+++ | | | | individual animals |
| | eVLPs | | Content) | | | | Y ELISpot** |
| 4 | Trivalent SARS- | 0.2 | E6020 plus Alum | | | | (Optional P1V014, P2Vd14) |
| | CoV-2, SRS-CoV | | (10 μg MPL + 33 μg | | | | |
| | and MERS-CoV | | of Al+++ Content) | | | | |
| | eVLPs | | (Aluminum Phosphate | | | | |

Each of the blood samples was tested for neutralizing antibodies at 14 days after first injection and 14 days after the second vaccination. As well, human serum from patients who had previously had confirmed cases of COVID-19 was also tested for neutralizing antibodies. Neutralizing antibodies were tested as follows. Vero cells were seeded in 6-well plates 48 h prior to infection. Sera was heat-inactivated at 56° C. for 30 min then quickly transfer on ice. Serum was diluted 1:10 with virus infection media then make serial dilutions of 1:2 for 8 subsequent dilutions. Equal volumes of diluted serum and virus (100 pfu per serum dilution) were mixed and incubated at 37° C. for 1 h. No sera and no virus controls were included. Cells were washed with PBS and each virus/serum were transferred and mixed to each well containing cells, and incubated at 37° C. for 1 h, with interval rocking of the plates. After the 1 h adsorption, excess inoculum was removed and a 2 ml virus infection media/agarose mix were overlaid onto the cells. The overlay was allowed to solidify and plates were incubated at 37° C. for 72 h. Cells were stained with crystal violet at 72 h post-infection. Plaques were quantified for all dilutions and PRNT titre was calculated. The % plaque reduction for all the dilutions based on the no serum control, was calculate using the Reed-Muench formula to determine the PRNT titre 90.

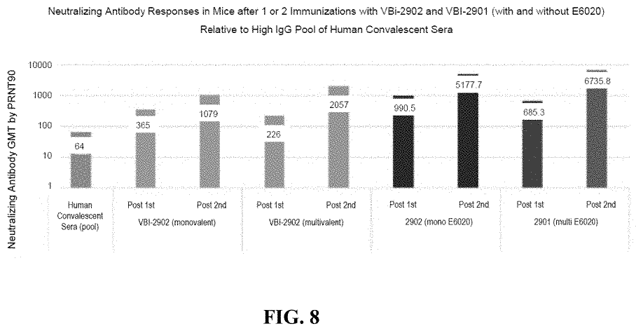

The results are shown in FIG. 8. The trivalent eVLP containing a pre-fusion version SARS-CoV-2 spike protein formulated with alum provided similar antibody titre to the monovalent eVLP vaccine containing the same prefusion spike protein formulated with alum (relative to non-pre-fusion prototypes). These results were significantly higher than the antibody titres observice in the serum from human convalescent patients. Specifically, after one dose of the alum-only eVLP vaccines, antibody titres were 3-6× higher than human convalescent serum levels. After two doses, the antibody titres were 15-20× higher than human convalescent levels. As is shown in FIG. 8, a single dose of the E6020 adjuvanted vaccines formulated with the trivalent and monovalent eVLP SARS-CoV-2 constructs produce 10-15× higher antibody titres when compared to the titres observed in human convalescent serum. Two doses of the E6020 adjuvanted eVLP vaccines produce ˜100 times higher antibody titres when compared to the antibody titres seen in human convalescent serum. Thus, one dose of E6020 adjuvanted vaccines produced antibody titres observed after two doses of alum-only vaccines.

### Example 9: Vaccination of Syrian Golden Hamsters with VBI-2902a and VBI-2902-e

Forty-eight (48) Syrian golden hamsters (males, aged approximately 5-6 weeks old) were purchased from Charles River Laboratories and were randomly assigned to Groups A, B, C and D (n=12/group, see Table 6). The randomly assigned group were administered with A. VBI-2902a (monovalent containing alum) vaccine, B. the VBI-2902e (monovalent containing alum and E6020) vaccine, C. a placebo, or D. a SARS-CoV-2 vaccine comprising the monovalent eVLP containing formulated with a proprietary adjuvant provided by the University of Saskatchewan (“TriAdj”). The monovalent eVLP vaccines contained the pre-fusion, stabilized version of the SARS-CoV-2 spike protein. The three components of the TriAdj per dose were 10 μg PCEP-3, 10 μg poly I:C and 20 μg IDR-1002. PCEP-3 was manufactured by Idaho National Laboratory. PolyI:C was purchased from Invivogen (Cat #+1r1-picw; Lot #PIW-11-03). IDR-1002 was synthesized by Peptide CPC Scientific (Cat #818360; Lot #CN-11-00590).

TABLE 6
Vaccine formulations and groupings for the vaccination of Syrian golden hamsters

| | | | | Adjuvant (μg/dose) | | | | |
|---|---|---|---|---|---|---|---|---|
| | | | | | Aluminium | | Route, | Injection |
| | | Vaccine | Antigen | | content | | Intramuscularly | Volume |
| Group | N | candidate | dose | E6020 | (Al+++) | TriAdj* | IM | (μl) |
| A | 12 | Placebo | 0 | NA | NA | NA | Intramuscularly | 100 μl |
| | | (Buffer 0.9% | | | | | via thigh (IM) | into |
| | | Saline) | | | | | | thigh |
| B | 12 | VBI-2902a | 1 μg | NA | 125 | NA | IM | 100 μl |
| | | (Monovalent | | | | | | into |
| | | SARS-CoV-2 | | | | | | thigh |
| | | Spike V4) | | | | | | |
| C | 12 | VBI-2902a | 1 μg | 10 | 33 | NA | IM | 100 μl |
| | | (Monovalent | | | | | | into |
| | | SARS-CoV-2 | | | | | | thigh |
| | | Spike V4) | | | | | | |
| D | 12 | SARS-CoV-2 | 1 μg | NA | NA | 10 μg poly | IM | 100 μl |
| | | Spike-V4 | | | | I:C, | | into |
| | | eVLP + TriAdj | | | | 20 μg IDR | | thigh |
| | | | | | | 10 μg PCEP | | |

*Triple adjuvant platform (TriAdj) consist of poly(I:C), innate defense regulator peptide (IDP) and poly[di(sodium carboxylatoethylphenoxy)]-phosphazene (PCEP)

The study duration was 56 days. Animals were acclimatized for 7 days before immunization. One day before immunization (Day −1), under anesthesia, a temperature transponder was implanted. The vaccines were administered twice at a 3-week interval, on Day 0, and Day 21, respectively. The vaccines were given via the intramuscular (IM) route at one side of the thighs, as specified in Table 5. The injection volume administered was 100 μL. On Day 42 (3 weeks after the boosting immunization), all animals were challenged with the SARS-CoV-2 virus intranasally with 50 μL/nare, via both nares. The challenge viral dose was 1×105 TCID50 per animal.

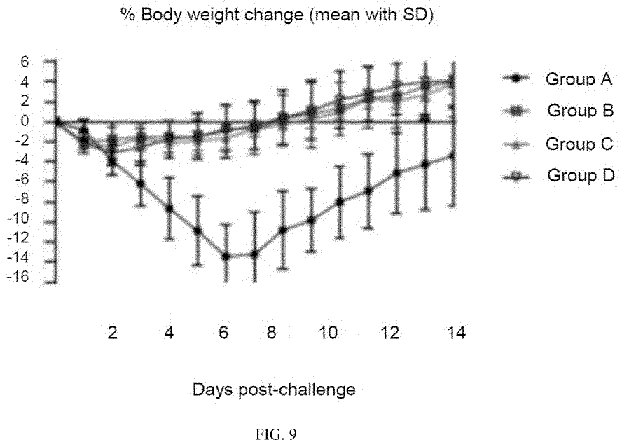

During the immunization phase, compared to the start day (Day0), hamsters gained approximately 10% weight at week 1, 20% at week 2 or 30% at week 3. All groups had similar patterns of body weight growth. After challenge, Group A animals (Saline controls) had body weight reduction and they lost body weights peaking at 6-8 days post-challenge (dpc), approximately 15% of the initial weights (FIG. 9). The medians of % body weight changes of the Group B, C or D animals were only about 1-2% and peaked at 2dpc (FIG. 9).

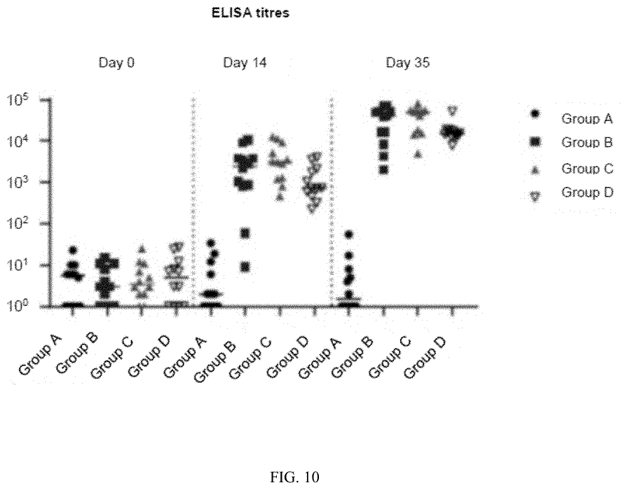

At Day 14, Group B or C animals produced antibody median titres of 2.9×103, and Group D at a median titre of 7.1×102 (FIG. 10). At Day 35, medians of antibody titres increased to 4.0×104 (Group B), 5.3×104 (Group C) and 1.7×104 (Group D). Antibodies for pre-bleed (prior to priming immunization) were at background levels for all animals. At Day 14 or Day 35, Group A animals did not increase antibody production.

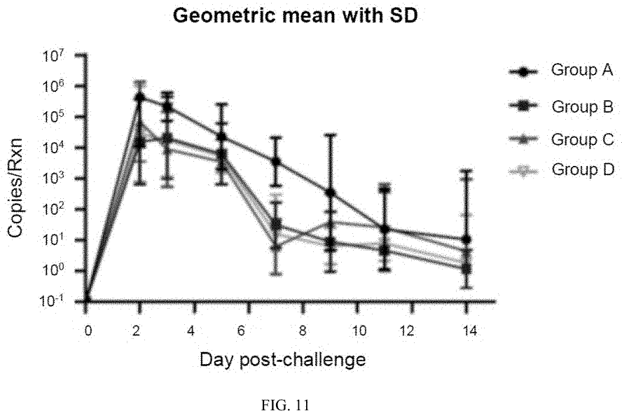

Viral RNA levels were at the peak at 2 day post-challenge (FIG. 11). Compared to Group A (Saline control), it seems that the levels of viral RNA were lower in Group B, C and D. Viral RNA levels were similar between Group B, C and D.

The above results from the Syrian golden hamster study further demonstrate that the vaccines of the present application are effective in preventing a SARS-CoV-2 infection.

All documents referenced in this disclosure are incorporated by reference herein, though if any incorporated document contradicts this written specification, then this written specification shall control. Those of skill in the will recognize that various changes and modifications may be made to the material provided herein, and that that material is within the scope and spirit of the disclosure.

We claim:

1. A vaccine comprising E6020 and virus-like particles comprising a coronavirus virus antigen and at least one additional adjuvant, wherein the at least one additional adjuvant is an aluminum-based adjuvant.

2. The vaccine according to claim 1, wherein the antigen is a SARS-CoV-2 spike protein possessing an amino acid sequence according to any one of SEQ ID NO: 1-4.

3. The vaccine according to claim 1, wherein the vaccine contains 0.1 μg to 100 μg of the antigen.

4. The vaccine according to claim 1, wherein the vaccine contains 1 μg to 25 μg of the antigen.

5. The vaccine according to claim 1, wherein the vaccine contains 5 μg to 10 μg of the antigen.

6. The vaccine according to claim 1, wherein the vaccine contains 1 μg to 50 μg of E6020.

7. The vaccine of claim 1, wherein the vaccine contains 0.5 μg to 20 μg of E6020.

8. The vaccine according to claim 1, wherein the virus-like particles comprise 0.05 mg to 0.5 mg of Gag protein.

9. The vaccine according to claim 1, wherein the vaccine contains 0.01 mg to 5 mg of the at least one additional adjuvant.

10. The vaccine according to claim 1, wherein the vaccine contains 0.05 mg to 1.0 mg of the at least one additional adjuvant.

11. The vaccine according to claim 1, wherein the vaccine contains 150 μg to 180 μg of the at least one additional adjuvant.

12. The vaccine according to claim 1, wherein the vaccine comprises 0.1 mg/mL to 0.9 mg/mL of the at least one additional adjuvant.

13. The vaccine according to claim 1, wherein the vaccine comprises 0.1 mg/mL to 0.5 mg/mL of the at least one additional adjuvant.

14. The vaccine according to claim 1, wherein the vaccine comprises 0.165 mg/mL to 0.33 mg/mL of the at least one additional adjuvant.

15. The vaccine according to claim 1, wherein the at least one additional adjuvant is selected from the group consisting of an aluminum hydroxide adjuvant, an aluminum phosphate adjuvant, an aluminum salt and any combination thereof.

16. The vaccine according to claim 1, wherein the at least one additional adjuvant is an aluminum phosphate adjuvant.

17. The vaccine according to claim 1, wherein said vaccine is formulated as a buffered solution.

18. The vaccine according to claim 1, wherein said vaccine is formulated as an emulsion.

19. The vaccine according to claim 18, wherein said emulsion comprises surfactants.

20. The vaccine according to claim 18, wherein said emulsion comprises polymers.

21. The vaccine according to claim 1, wherein the vaccine is formulated as microparticles or nanoparticles.

22. A method of generating an immune response in a subject in need of mitigation of coronavirus infection comprising administering to the subject a vaccine according to any one of claims 1-21.

23. The method according to claim 22, wherein said vaccine is administered intranasally, intravenously, intradermally, intramuscularly or subcutaneously.

24. The method according to claim 22, wherein the vaccine is administered in a dose ranging from 0.1 mL to 10 mL.

25. The method according to claim 22, wherein the vaccine is administered in a dose ranging from 0.2 mL to 5 mL.

26. The method according to claim 22, wherein the vaccine is administered in a dose ranging from 0.3 mL to 3.0 mL.

27. A vaccine comprising E6020 and virus-like particles comprising an antigen.

28. The vaccine according to claim 27, wherein the antigen is derived from an influenza or corona virus.

29. The vaccine according to claim 27, wherein the antigen is a SARS-CoV-2 spike protein possessing an amino acid sequence according to any one of SEQ ID NO: 1-4.

30. The vaccine according to claim 27, wherein the vaccine contains 0.1 μg to 100 μg of the antigen.

31. The vaccine according to claim 27, wherein the vaccine contains 1 μg to 25 μg of the antigen.

32. The vaccine according to claim 27, wherein the vaccine contains 5 μg to 10 μg of the antigen.

33. The vaccine according to claim 27, wherein the vaccine contains 1 μg to 50 μg of E6020.

34. The vaccine according to claim 27, wherein the vaccine contains 0.5 μg to 10 μg of E6020.

35. The vaccine according to claim 27, wherein the vaccine contains 0.1 μg to 100 μg of E6020.

36. The vaccine according to claim 27, wherein the vaccine contains 0.5 μg to 50 μg μg of E6020.

37. The vaccine according to claim 27, wherein the virus-like particles comprise a Gag protein of murine leukemia virus.

38. The vaccine according to claim 27, wherein the vaccine comprises at least one additional adjuvant selected from the group consisting of an aluminum hydroxide adjuvant, an aluminum phosphate adjuvant, an aluminum salt, chemokines, cytokines, nucleic acid sequences, lipoprotein, lipopolysaccharide, monophosphoryl lipid A, lipoteichoic acid, imiquimod, reiquimod, QS-21 and any combination thereof.

39. The vaccine according to claim 38, wherein the vaccine comprises 50 μg to 50 mg of the at least one additional adjuvant.

40. The vaccine according to claim 38, wherein the vaccine comprises 0.1 mg to 20.0 mg of the at least one additional adjuvant.

41. The vaccine according to claim 38, wherein the vaccine comprises 1.0 mg to 5.0 mg of the at least one additional adjuvant.

42. The vaccine according to claim 38, wherein the at least one additional adjuvant is an aluminum hydroxide adjuvant or an aluminum phosphate adjuvant.

43. The vaccine according to claim 27, wherein the virus-like particles comprise 10 μg to 10 mg of Gag protein.

44. The vaccine according to claim 27, wherein the virus-like particles contain 50 μg to 5 mg of Gag protein.

45. The vaccine according to claim 27, wherein the virus-like particles contain 100 μg to 500 μg of Gag protein.

46. The vaccine according to claim 27, wherein the virus-like particles contain 1 μg to 50 mg of Gag protein.

47. The vaccine according to claim 27, wherein the vaccine is formulated as a buffered solution.

48. The vaccine according to claim 27, wherein the vaccine is formulated as an emulsion.

49. The vaccine according to claim 48, wherein said emulsion comprises surfactants.

50. The vaccine according to claim 48, wherein said emulsion comprises polymers.

51. A method of generating an immune response in a subject in need of mitigation of coronavirus infection comprising administering to the subject a vaccine according to any one of claims 27-50.

52. The method according to claim 51, wherein said vaccine is administered intranasally, intravenously, intradermally, intramuscularly or subcutaneously.

53. The method according to claim 51, wherein the vaccine is administered in a dose ranging from 0.1 mL to 10 mL.

54. The method according to claim 51, wherein the vaccine is administered in a dose ranging from 0.2 mL to 5 mL.

55. The method according to claim 51, wherein the vaccine is administered in a dose ranging from 0.3 mL to 3.0 mL.

56. A vaccine comprising E6020 and a Nidovirales virus antigen.

57. The vaccine of claim 56, wherein the Nidovirales virus antigen is a coronavirus antigen.

58. The vaccine of claim 56, wherein the coronavirus antigen is a SARS-CoV-2 antigen.

59. The vaccine according to claim 56, further comprising one or more additional adjuvants.

60. The vaccine according to claim 56, wherein said vaccine is formulated as a buffered solution.

61. The vaccine according to claim 56, wherein said vaccine is formulated as an emulsion.

62. The vaccine according to claim 61, wherein said emulsion comprises surfactants.

63. The vaccine according to claim 61, wherein said emulsion comprises polymers.

64. The vaccine according to claim 56, wherein said vaccine is formulated as microparticles or nanoparticles.

65. A method of generating an immune response in a subject in need of mitigation of coronavirus infection comprising administering to the subject a vaccine according to any one of claims 56-64.

66. The method according to claim 65, wherein said vaccine is administered intranasally, intravenously, intradermally, intramuscularly or subcutaneously.

67. The method according to claim 65, wherein the vaccine is administered in a dose ranging from 0.1 mL to 10 mL.

68. A method of generating an immune response in a subject in need of mitigation of coronavirus infection comprising administering to the subject a vaccine comprising E6020 and an antigen.

69. The method according to claim 68, wherein the antigen is derived from a viral or bacterial pathogen.

70. The method according to claim 68, wherein the antigen is derived from the influenza virus.

71. The method according to claim 68, wherein the antigen is derived from the corona virus.

72. The method according to claim 68, wherein the antigen is a SARS-CoV-2 spike protein.

73. The method according to claim 68, wherein the vaccine further comprises one or more additional adjuvants.

74. The method according to claim 68, wherein the vaccine is formulated as a buffered solution.

75. The method according to claim 68, wherein said vaccine is formulated as an emulsion.

76. The method according to claim 75, wherein said emulsion comprises surfactants.

77. The method according to claim 75, wherein said emulsion comprises polymers.

78. The method according to claim 69, wherein said vaccine is formulated as microparticles or nanoparticles.

79. The method according to claim 69, wherein said vaccine is administered intranasally, intravenously, intramuscularly or subcutaneously.

80. The method according to claim 69, wherein the vaccine is administered in a dose ranging from 0.1 mL to 10 mL.
